(19) United States
(12) Patent Application Publication
(10) Pub. No.: US 2021/0252644 A1
(43) Pub. Date: Aug. 19, 2021

(54) pH ELECTRODE WITH CARBON REGION

(71) Applicant: Hach Company, Loveland, CO (US)

(72) Inventor: Zoë Duncan, Daventry (GB)

(21) Appl. No.: 17/126,730

(22) Filed: Dec. 18, 2020

Related U.S. Application Data
(60) Provisional application No. 62/976,043, filed on Feb. 13, 2020.

(51) Int. Cl. B23K 26/359; G01N 27/30; B23K 26/0622; B23K 26/402

(52) U.S. Cl. CPC B23K 26/359; G01N 27/302; B23K 2101/36; B23K 26/0622; B23K 26/402; G01N 27/308

(57) ABSTRACT

An embodiment provides a method for manufacturing a pH electrode with a carbon region, including: determining at least one pulse overlap modification for a laser pulse used when machining the carbon region; selecting a pattern for machining the carbon region; and machining a sp2 carbon region having the at least one pulse overlap modification and in the selected pattern by pulsing a laser onto a BDD electrode surface in the selected pattern with the at least one pulse overlap modification. Other aspects are described and claimed.

### CROSS REFERENCE TO RELATED APPLICATION

This application claims priority to U.S. Provisional Patent Application Ser. No. 62/976,043, filed on Feb. 13, 2020, and entitled “pH ELECTRODE WITH CARBON REGION,” the contents of which are incorporated by reference herein.

### FIELD

This application relates generally to pH measurement of an aqueous sample, and, more particularly, to pH electrodes with a carbon region.

### BACKGROUND

Ensuring water quality is critical to the health and well-being of humans, animals, and plants, which are reliant on water for survival. One parameter of water that may be measured is the pH. The measurement of pH of an aqueous sample is critical in a number of industries such as pharmaceuticals, biomedical, water supply, and other manufacturing fields. Measurement of pH may allow for proper treatment of water or ensuring proper water quality for sensitive purposes, and allows for identifying the overall quality of the water. One method to measure pH in an aqueous sample includes the use of electrodes which require constant maintenance and calibration of the pH measurement system.

### BRIEF SUMMARY

In summary, one embodiment provides a method for manufacturing a Boron Doped Diamond (BDD) pH electrode with a carbon region, comprising: determining at least one pulse overlap modification for a laser pulse used when machining the carbon region; selecting a pattern for machining the carbon region; and machining a sp2 carbon region having the at least one pulse overlap modification and in the selected pattern by pulsing a laser onto a BDD electrode surface in the selected pattern with the at least one pulse overlap modification.

Another embodiment provides a device for laser machining a pH electrode with a carbon region, comprising: a machining instrument having a laser providing a laser pulse; a processor; a memory device that stores instructions executable by the processor to: determine at least one pulse overlap modification for a laser pulse used when machining the carbon region; select a pattern for machining the carbon region; and machine a sp2 carbon region having the at least one pulse overlap modification and in the selected pattern by pulsing a laser onto a BDD electrode surface in the selected pattern with the at least one pulse overlap modification.

A further embodiment provides a system for laser machining a pH electrode with a carbon region, comprising: a machining device having a laser providing a laser pulse; a BDD electrode comprising a BDD electrode surface; and a storage device having code stored therewith, the code being executable by the processor and comprising: code that determines at least one pulse overlap modification for a laser pulse used when machining the carbon region; code that selects a pattern for machining the carbon region; and code that machines a sp2 carbon region having the at least one pulse overlap modification and in the selected pattern by pulsing a laser onto the BDD electrode surface in the selected pattern with the at least one pulse overlap modification.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1A illustrates a schematic diagram of pulse overlap in an example embodiment. FIG. 1B illustrates a schematic diagram of another pulse overlap in an example embodiment. FIG. 1C illustrates a schematic diagram of a further pulse overlap in an example embodiment.

FIG. 2A illustrates a schematic diagram of a pattern in an example embodiment. FIG. 2B illustrates a schematic diagram of another pattern in an example embodiment. FIG. 2C illustrates a schematic diagram of a further pattern in an example embodiment.

FIG. 3A illustrates a schematic diagram of another pattern in an example embodiment. FIG. 3B illustrates a schematic diagram of another pattern in an example embodiment. FIG. 3C illustrates a schematic diagram of a further pattern in an example embodiment.

FIG. 4 illustrates an example of computer circuitry.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

The measurement of the pH of water or other aqueous solutions or samples is common and allows for determination of the quality or other characteristics of the aqueous solution. Conventional pH electrodes for measurement of pH may be constructed using fragile, thin glass. This glass breaks easily leading to higher replacement and maintenance costs. The possible breakage of glass pH electrodes may also limit their use in food and beverage applications. Conventional pH electrodes also may have “alkali errors.” These errors arise from interfering ions such as sodium and potassium affecting the pH response at high pH values. A commercial need exists for a robust pH measurement electrode that requires less maintenance while maintaining measurement of pH in sample containing heavy metals or a low conductivity sample.

Another method of measuring pH of an aqueous sample uses a laser machined boron-doped diamond (BDD) material to create a sensor capable of measuring pH. Laser machining of the BDD creates pH sensitive quinone-like structures on the BDD material, due to the introduction of sp2 carbon. These laser machined areas or pits may be in an array pattern. In other words, the laser machined areas may be a series of spots across a surface. The laser machining of these multiple spots may be challenging. For example, an array of laser machines pits across a surface requires using the laser repeatedly across the surface. Repeating machining of a BDD surface with a laser may create heat. The generation of heat may lead to further creation of sp2 carbon, and subsequent quinone-like structures capable of sensing pH. Repeated laser machining also requires laser pulses which may be time consuming in the manufacturing process. A proper control of a laser machining may maintain a proper aspect ratio of features to prevent fouling or blocking of an electrode surface.

A BDD electrode may give greater accuracy, especially improved accuracy in samples with low conductivity or low buffer capacity. BDD electrodes may measure pH in all pH ranges including an environmentally relevant range of pH 4 to 11. A better control over quinone-like structure generation is required as the quinone-like structures may impact accuracy in low conductive and low buffer capacity solutions. Heating from a laser machining process may lead to greater quinone-like structures. What is needed is a method and system to control production of quinone-like structures and/or reduce heating during a laser machining. Additionally, the system and method may reduce manufacturing time.

Accordingly, the systems and methods as described herein may be used to make an electrode. The electrode may be a BDD electrode. The electrode may be sensitive to pH. The pH sensitive region may be laser machined. The laser machining may create a carbon region. The carbon region may be a sp2 carbon region. The carbon region may have oxidized carbon structures and or quinone-like groups. In an embodiment, the system and method may determine a pulse overlap modification. A pulse overlap modification may refer to the manner in which laser pulses overlap or do not overlap. The pulse overlap modification may be 2-dimensional, 1-dimensional, and/or 0-dimensional, as described below. In an embodiment, the method and system may select a pattern for machining a carbon region. For example, the pattern may be an array, parallel line, lines, concentric circle, or the like. Other patterns are described and disclosed. A laser machined electrode may determine a pH by identifying an electrical potential of an aqueous sample.

The electrode may have a carbon region and a pH sensitive carbon region. For example, the primary electrode may be a BDD electrode with a plurality of sp2 carbon regions. The primary electrode may be a working electrode. The pH sensitive carbon region may be laser machined. The carbon region may comprise sp2 carbon materials that can include diamond-like materials doped with elements like boron (BDD). The pH sensitive carbon region may be an sp2 carbon region that is included on a boron doped diamond-based pH electrode. Being included may mean that the sp2 carbon region is introduced into, integrated into, contained within, laser micro machined into, or otherwise integrated into the boron doped diamond electrode. In other words, while the sp2 carbon region and the boron doped diamond are integrated into the same electrode, they are chemically different regions of the electrode. The carbon region may have oxidized carbon structures. The oxidized carbon structure may have quinone or quinone-like groups.

The electrode produced by a described method and system may also be used with at least one reference electrode and at least one auxiliary electrode. In an embodiment, an applied potential protocol may be applied to a first measurement electrode. An electrical potential between the measurement electrode and a reference electrode may be measured. In an embodiment, the electrical potential may correspond to a pH measurement of an aqueous sample. The measurement electrode, at least one reference electrode, and at least one auxiliary electrode may be operatively coupled to circuitry to measure, analyze, and store pH measurements of a sample.

The use of BDD serves as a better electrode material than other carbon-based or metallic materials (e.g., silver, gold, mercury, nickel, etc.) because these materials may be more electrocatalytically active, and may generate interfering signals contributing to the errors in the measurement of pH.

The illustrated example embodiments will be best understood by reference to the figures. The following description is intended only by way of example, and simply illustrates certain example embodiments.

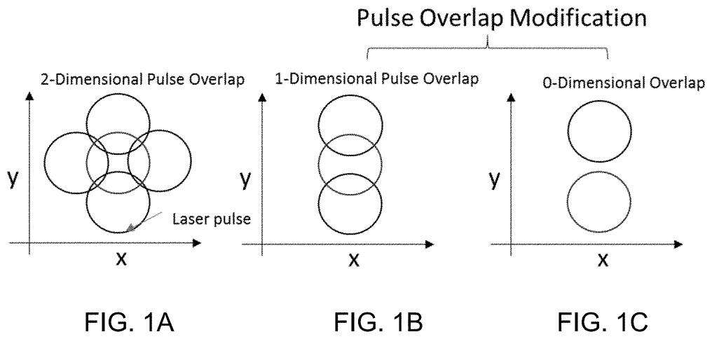

Referring now to FIG. 1, an embodiment may determine a pulse overlap modification for an electrode. The pulse overlap medication may refer to the method in which an electrode is laser machined or laser pulsed to produce a functional area upon the electrode face. In an embodiment, the electrode may be a BDD electrode. BDD electrode may reduce fragility, alkali errors, wet storage requirements, or the like as compared to glass electrode.

In an embodiment, the electrode may measure pH of a sample. In an embodiment, an electrode may be laser micromachined or machined to introduce an array of pits into the electrode surface or face. In an embodiment, a combination of pits may create a pattern upon the electrode. The laser machining may introduce sp2 carbon upon the electrode. The sp2 carbon may include quinone or quinone-like groups which may undergo proton-coupled electron transfer. In an embodiment, the laser machined electrodes may be used to perform electrochemical measurements to measure a pH response on the electrode in which the observed peak may be indicative of a pH of a sample.

In an embodiment, a BDD pH electrode may operate in a Nernstian manner across a pH range in a buffered solution. In an unbuffered solution, a BDD pH electrode may deviate from an expected theoretical response. A deviation may be linked to an amount of sp2 carbon present and/or the amount of quinone-like groups on an electrode surface. Thus, control of carbon region machining may be critical to the efficient manufacture of an accurate electrode. The system and method disclosed may provide better manufacturing of a suitable BDD pH electrode with sp2 regions.

In an embodiment, a pulse overlap modification may be determined to laser machine a BDD pH electrode. In an embodiment, a 2-dimensional machining process may be used to manufacture a BDD pH electrode. A 2-dimensional machining may be characterized when three or more laser pulses overlap. A 2-dimensional machining may result in a larger area of graphitization. A 2-dimensional machining may result in a creation of sp2 carbon within a BDD surface. A 2-dimensional machining may produce sites upon the BDD that are machined more than once (re-machining). Re-machining may lead to an increase of sp2 carbon, and may create a different distribution of quinone as compared to a single pulse or single laser machining. Re-machining before cooling down of the electrode surface may lead to the creation of more sp2 carbon. Re-machining may be inefficient and leads to longer manufacturing times.

In an embodiment, a different pulse overlap modification may be determined. In other words, laser machining parameters may be modified such that pulse overlap, which may be referred to as pitch, is adjusted. In an embodiment, the pulse overlap may be adjusted such that a single spot on the surface is machined no more than twice. For example, an area may be laser machined with a 1-dimensional pulse overlap modification. A 1-dimensional pulse overlap modification may be used to laser machine a line, hash, circle, linear shapes that may be rastered, or the like.

In an embodiment, a 0-dimensional pulse overlap may be used to manufacture an electrode. In other words, a 0-dimensional pulse overlap may have no overlap of laser pulses. A 0-dimensional overlap may allow a laser machining method to machine a surface only once. In other words, the laser machining of an sp2 carbon region into the BDD surface may be controlled.

Laser machining may heat the electrode surface. A pulse overlap may be selected to minimize or mitigate the heating of an electrode surface. A determination of a pulse overlap may also facilitate a faster and/or more controlled laser machining process. For example, less pulse overlap may reduce multiple laser pulses over one area of an electrode and allow laser machining in a more efficient manner.

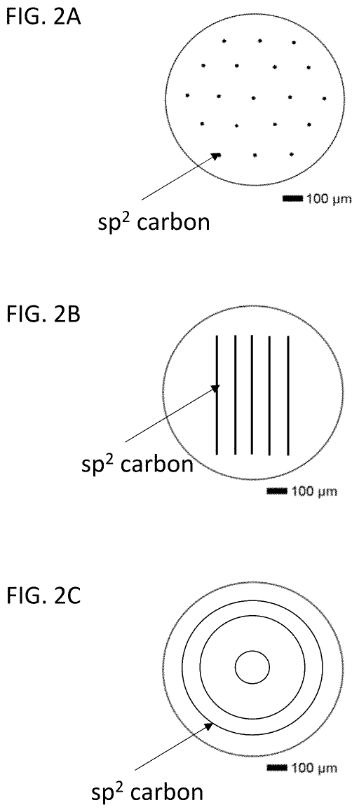

Referring to FIG. 2, example embodiments of a pattern may be selected. A pattern may refer to the shape or pattern of laser machined areas upon the electrode surface. A pattern may refer to an array, one or more lines, circles, or the like. In other words, a BDD electrode may be laser machined in one or more areas to produce a sp2 carbon area. The sp2 carbon area may be functionalized to be pH sensitive, as an example. In an embodiment, an array may be laser machined (FIG. 2A). An array may be a plurality of laser machined spots, areas, pits, or the like across an electrode surface. The plurality of laser machined areas may be of a uniform diameter, varying diameter, evenly spaced, spaced, or the like based upon an electrode application or use.

In an embodiment, at least one line may be laser machined (FIG. 2B). In an embodiment there may be one line or two or more lines laser machined. The lines may be parallel or non-parallel. The lines may be a uniform or different length and/or width. The laser machining may not be a straight line. For example, the laser machining may be a curved section, S-shaped, geometric shapes, or the like.

In an embodiment, a circle or oval like shape may be laser machined (FIG. 2C). In an embodiment, the circles or oval may be concentric or non-concentric. The spacing and placement of the circles or ovals may be uniform or different. In an embodiment, any combination of laser machining upon an electrode may be performed. The examples are illustrative, and other embodiments are disclosed.

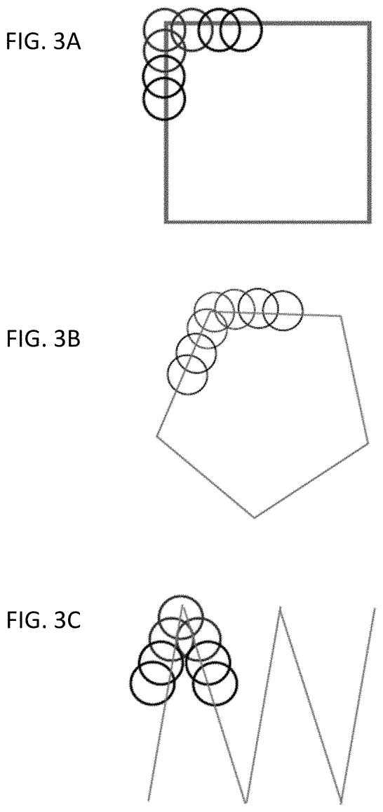

Referring to FIG. 3, example embodiments of a pulse overlap and a pattern are illustrated. For example, a laser machining of a square (FIG. 3A), a pentagon (FIG. 3B), and a zig-zag (FIG. 3C) pattern are illustrated. In an embodiment, pulse overlap modification may be used to reduce the re-machining using laser pulses upon the electrode. In an embodiment, despite a reduction of pulse overlap, there may still be pulse overlap. In an embodiment, pulse overlap may occur in a location such as a corner of a geometric shape. For example, is a system determines a 1-dimensional pulse overlap, a 2-dimensional pulse overlap may occur as the machining process changes direction such as turning a corner. As another example, a more acute angle on a shape may create a greater pulse overlap as compared to an obtuse angle of a shape. Thus, while an embodiment may prefer a 1-dimensional or 0-dimensional pulse overlap, a greater pulse overlap may occur at some areas. In an embodiment, this greater pulse overlap may be a very small amount of area as compared to the entire lase machined surface area upon the electrode. In an embodiment, the system may reduce laser pulses in a corner, bend, turn, or the like to reduce pulse overlap.

In an embodiment, the laser machining may be tailored for a specific application. For example, pulse overlap may be reduced to reduce a heating of the electrode material. Heat may cause damage of an electrode, reduction of tolerances, reduction of accuracy, or the like. In an embodiment, the laser machining may be adjusted. Parameters that may be adjusted include, but are not limited to: size, diameter, number, size, shape, length of time of machining, speed, depth, or the like of a laser pulse. In an embodiment, a laser machining may employ different parameters on the same or different electrodes.

The method and system may control the laser machining process. For example, the system may control the x-y coordinates of the laser pulse upon an electrode, the length of time of a pulse, speed of movement of the laser, latency between pulses, pulse overlap, pattern type, diameter, power, depth, or the like. The system may laser machine one or more electrodes either sequentially or in parallel. The system may be preprogrammed either from an input of instruction by a user or from a stored database upon the device, cloud, or the like. The system may adjust parameters to fit the needs of a user, application, quality control protocols, or the like.

In an embodiment, the laser machined electrode may be used for a pH measurement, interferent measurement, electrochemical measurement, or the like. The system may have at least one reference electrode. The system may have at least one auxiliary electrode. In an embodiment, the system may comprise an electrode that is a BDD electrode with at least one sp2 carbon region laser machined upon a face of the electrode. In an embodiment, the method and system may comprise an additional electrode. The additional electrode may be a BDD electrode with no sp2 region or an sp2 region with a lesser area than the sp2 area of a primary electrode. In an embodiment, the laser machined electrode may be a part of a 3-electrode system. For example, the system may measure using an auxiliary, a reference, and either a primary or secondary electrode.

The laser machined electrode may also be used with at least one reference electrode and one auxiliary electrode. In an embodiment, an applied potential protocol may be utilized and a resultant electrical potential may be measured between the at least one measurement electrode and at least one reference electrode. The systems and methods as described herein provide a technique for accurate measurement of pH in a range of sample types such as samples with low buffer capacity or samples with low conductivity.

In an embodiment, the carbon region may be of a BDD material, BDD sp2 material, quinone structures, quinone-like structures, oxidized carbon structures, or the like. The carbon material may be laser machined upon the measurement electrode. The measurement electrode may be a BDD electrode. The laser machining may be a plurality of laser machined areas or a continuous region. The laser machined region may be circular or circular-like in shape such as a circle, oval, or the like. In other words, other shapes of carbon regions are disclosed and may be laser machined to a particular use or configuration of a measurement electrode.

In an embodiment, the size, depth, or surface area of the laser machined sp2 region may be constructed for a particular use. In other words, the sp2 region may be machined in accordance with actual or expected pH ranges, conductivity range, buffer capacity, heavy metal type, heavy metal concentration, size of particles or components, or the like within a sample to be measured. In an embodiment, the sp2 carbon regions may be of a different number and/or diameter. For example, the diameter of a single sp2 laser machined area may be larger or smaller. As another example, the number of sp2 regions may be increased or decreased on a given electrode. The diameter and number of sp2 regions may be increased or decreased upon an electrode.

In an embodiment, the laser machined electrodes may be fully or at least partially disposed in the volume of aqueous solution. For example, if the aqueous solution is introduced into a chamber having one or more electrodes, the aqueous solution may at least partially cover the one or more electrodes. As another example, the one or more electrodes may be partially disposed within the chamber with the other portion of the electrode outside the chamber. Thus, when the aqueous solution is introduced into the chamber it only covers the portion of the electrodes that are within the chamber.

The laser machined electrode may measure an electrical potential of the volume of aqueous sample across at least one measurement electrode and at least one reference electrode. A measurement system may have either, one measurement electrode and one reference electrode, or have a plurality of measurement electrodes and a plurality of reference electrodes.

The electrical potential may be measured across one or more electrodes, for example, a series of electrodes. In an embodiment, the primary measurement electrode may be used to measure the electrical potential attributable to a pH of an aqueous sample. The measurement electrode may contain a carbon region. The carbon region is described above. The carbon region may be laser machined. In an embodiment, the carbon region may be a BDD, BDD sp2, oxidized carbon structure, quinone, quinone-like structure or the like. The carbon region may be a continuous region in a circular-like shape, a line, a geometric shape, or the like.

The various embodiments described herein thus represent a technical improvement to conventional methods and instrument for pH measurement. Using the techniques as described herein, an embodiment may use a method and device for an instrument for pH measurement. This is in contrast to conventional methods with limitations mentioned above. Such techniques provide a better method to construct an instrument for pH measurement.

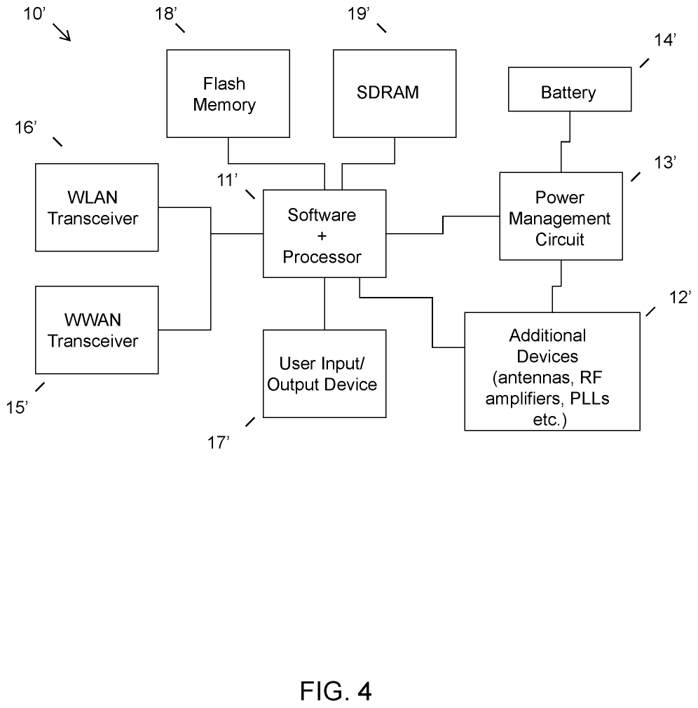

While various other circuits, circuitry or components may be utilized in information handling devices, with regard to an instrument for pH measurement according to any one of the various embodiments described herein, an example is illustrated in FIG. 4. Device circuitry 10′ may include a measurement system on a chip design found, for example, a particular computing platform (e.g., mobile computing, desktop computing, etc.) Software and processor(s) are combined in a single chip 11′. Processors comprise internal arithmetic units, registers, cache memory, busses, I/O ports, etc., as is well known in the art. Internal busses and the like depend on different vendors, but essentially all the peripheral devices (12′) may attach to a single chip 11′. The circuitry 10′ combines the processor, memory control, and I/O controller hub all into a single chip 11′. Also, systems 10′ of this type do not typically use SATA or PCI or LPC. Common interfaces, for example, include SDIO and I2C.

There are power management chip(s) 13′, e.g., a battery management unit, BMU, which manage power as supplied, for example, via a rechargeable battery 14′, which may be recharged by a connection to a power source (not shown). In at least one design, a single chip, such as 11′, is used to supply BIOS like functionality and DRAM memory.

System 10′ typically includes one or more of a WWAN transceiver 15′ and a WLAN transceiver 16′ for connecting to various networks, such as telecommunications networks and wireless Internet devices, e.g., access points. Additionally, devices 12′ are commonly included, e.g., a transmit and receive antenna, oscillators, PLLs, etc. System 10′ includes input/output devices 17′ for data input and display/rendering (e.g., a computing location located away from the single beam system that is easily accessible by a user). System 10′ also typically includes various memory devices, for example flash memory 18′ and SDRAM 19′.

It can be appreciated from the foregoing that electronic components of one or more systems or devices may include, but are not limited to, at least one processing unit, a memory, and a communication bus or communication means that couples various components including the memory to the processing unit(s). A system or device may include or have access to a variety of device readable media. System memory may include device readable storage media in the form of volatile and/or nonvolatile memory such as read only memory (ROM) and/or random access memory (RAM). By way of example, and not limitation, system memory may also include an operating system, application programs, other program modules, and program data. The disclosed system may be used in an embodiment of an instrument for pH measurement.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device, where the instructions are executed by a processor. In the context of this document, a storage device is not a signal and “non-transitory” includes all media except signal media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and products according to various example embodiments. It will be understood that the actions and functionality may be implemented at least in part by program instructions. These program instructions may be provided to a processor of a device, e.g., a measurement device such as illustrated in FIG. 1, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for manufacturing a pH electrode with a carbon region, comprising:
determining at least one pulse overlap modification for a laser pulse used when machining the carbon region;
selecting a pattern for machining the carbon region; and
machining a sp2 carbon region having the at least one pulse overlap modification and in the selected pattern by pulsing a laser onto a BDD electrode surface in the selected pattern with the at least one pulse overlap modification.

2. The method of claim 1, wherein the pulse overlap modification is a 1-dimensional overlap.

3. The method of claim 1, wherein the pulse overlap modification is a 0-dimensional overlap.

4. The method of claim 1, wherein the pattern is a plurality of spots upon the BDD electrode surface.

5. The method of claim 1, wherein the pattern is a plurality of parallel lines upon the BDD electrode surface.

6. The method of claim 1, wherein the pattern is a plurality of concentric circles.

7. The method of claim 1, wherein the determining at least one pulse overlap modification is based upon the pattern selected.

8. The method of claim 1, wherein the carbon region of the BDD electrode surface is pH sensitive.

9. The method of claim 1, wherein the carbon region of the measurement electrode comprises at least one of: oxidized carbon structures and quinone-like groups.

10. The method of claim 1, wherein the at least one pulse overlap modification is determined to reduce an amount of sp² carbon into the BDD electrode surface.

11. A device for laser machining a pH electrode with a carbon region, comprising:
a machining instrument having a laser providing a laser pulse;
a processor;
a memory device that stores instructions executable by the processor to:
determine at least one pulse overlap modification for a laser pulse used when machining the carbon region;
select a pattern for machining the carbon region; and
machine a sp2 carbon region having the at least one pulse overlap modification and in the selected pattern by pulsing a laser onto a BDD electrode surface in the selected pattern with the at least one pulse overlap modification.

12. The device of claim 11, wherein the pulse overlap modification is a 1-dimensional overlap.

13. The device of claim 11, wherein the pulse overlap modification is a 0-dimensional overlap.

14. The device of claim 11, wherein the pattern is a plurality of spots upon the BDD electrode surface.

15. The device of claim 11, wherein the pattern is a plurality of parallel lines upon the BDD electrode surface.

16. The device of claim 11, wherein the pattern is a plurality of concentric circles.

17. The device of claim 11, wherein the determining at least one pulse overlap modification is based upon the pattern selected.

18. The device of claim 11, wherein the carbon region of the BDD electrode surface is pH sensitive.

19. The device of claim 11, wherein the carbon region of the measurement electrode comprises at least one of: oxidized carbon structures and quinone-like groups.

20. A system for laser machining a pH electrode with a carbon region, comprising:
a machining device having a laser providing a laser pulse;
a BDD electrode comprising a BDD electrode surface; and
a storage device having code stored therewith, the code being executable by the processor and comprising:
code that determines at least one pulse overlap modification for a laser pulse used when machining the carbon region;
code that selects a pattern for machining the carbon region; and
code that machines a sp2 carbon region having the at least one pulse overlap modification and in the selected pattern by pulsing a laser onto the BDD electrode surface in the selected pattern with the at least one pulse overlap modification.
